(19) United States
(12) Patent Application Publication
(10) Pub. No.: US 2014/0339467 A1
(43) Pub. Date: Nov. 20, 2014

(54) HIGH SURFACE AREA COMPOSITION COMPRISED OF LIGNIN

(71) Applicant: Biochemtex S.p.A., Tortona (IT)

(72) Inventors: Guliz Arf Elliott, Akron, OH (US); Danilo De Faveri, Novi Ligure (IT); Francesco Cherchi, Novi Ligure (IT); Simone Ferrero, Tortona (IT); Paolo Torre, Arenzano (IT)

(21) Appl. No.: 14/363,566

(22) PCT Filed: Dec. 20, 2012
(86) PCT No.: PCT/EP2012/076439

§ 371 Date: Jun. 6, 2014

Related U.S. Application Data
(60) Provisional application No. 61/578,373, filed on Dec. 21, 2011.
(60) Provisional application No. 61/736,649, filed on Dec. 13, 2012.

(30) Foreign Application Priority Data
Jan. 11, 2012 (IT) TO2012A000014

(51) Int. Cl. C08B 1/00; C12P 19/02; C12P 19/14

(52) U.S. Cl. CPC C08B 1/003; C12P 19/14; C12P 19/02
USPC 252/182.12; 435/99

(57) ABSTRACT

Disclosed in this specification is a lignin composition having unique characteristics relative to its characteristics as found in its natural environment. The lignin has been modified so that more lignin decomposes at the lower lignin decomposition temperature than decomposes at the higher lignin decomposition temperature and the lignin composition has a very high surface area relative to naturally occurring lignin compositions.

### BACKGROUND

In “Thermal degradation of lignin—a review”, Brebu at al, Cellulose Chem. Technol., 44 (9), page 353-363 (2010), the authors report that lignin decomposes slower, over a broader temperature range (200-500° C.) than cellulose and the hemicellulose components of biomass. Degradation studies performed on different types of lignin by thermal analysis (DTA) showed an endothermic peak at 100-180° C., corresponding to the elimination of humidity, followed by two broad exothermal peaks, the first one from 280 to 390° C. and the second one at higher temperatures, with a peak around 420° C. and a long tail beyond 500° C. The DTG curves of lignin decomposition show wide and flat peaks with a gently sloping baseline that makes it impossible to define an activation energy for the reaction. This is different for the sharper DTG peaks of cellulose and hemicellulose, inducing a flat tailing section at higher temperatures for wood decomposition.

The authors note also that, due to its complex composition and structure, the degradation of lignin is strongly influenced by its nature, reaction temperature, heating rate and degradation atmosphere, which also affects the temperature domain of degradation, conversion and product yields.

In “Lignin Changes after Steam Explosion and Laccase-Mediator Treatment of Eucalyptus Wood Chips”, Martin-Sampedro et al., J. Agric. Food Chem. 2011, 59, 8761-8769, thermogravimetric analyses were used to characterize the different wood chip samples. The analysis was based on the fact that each of the fiber cell wall polymers has a distinctive degradation temperature and rate of energy release upon thermal breaking and combustion. During TGA the ligno-cellulosic samples were kept under air atmosphere, and two main temperature ranges of degradation were observed, 250-350 and 400-500° C. These are attributed to degradation of polysaccharides and lignin, respectively. Relative to cellulose and hemicelluloses, which are aliphatic structures, the higher degradation temperature for lignin is ascribed to its aromatic structure.

In “Lignin—a useful bioresource for the production of sorption-active materials” Dizhbite et al., Bioresource Technology 67 (1999) 221-228, different compositions derived from ligno-cellulosic materials are presented. In Table 2, the specific area of compositions ranges from 84 m²/g to 601 m²/g and the lignin content varies between 51.2% and 97.8%.

The surface area of lignin and lignin chars can be found in “Lignin—from natural adsorbent to activated carbon: A review”, Carrott and Carrott, Bioresource Technology 98 (2007) 2301-2312. This article compiles the work done over the last few decades on the use of lignin and lignin-based chars. It reports a BET in m²/g of the char of olive waste and untreated wheat straw as 3.1 and 68.7 respectively. In general, the literature tabulated by the article reports lignin char as having a surface area greater than 500 m²/g (Table 3 of Article). Char is the solid material that remains from a carbonaceous material after a process of combustion. In “Effect of steam explosion on biodegradation of lignin in wheat straw”, Zhang et al., Bioresource Technology 99 (2008) 8512-8515, the authors compare surface morphology of biodegraded raw material (BRM) and biodegraded raw material after steam explosion (BSE), and noted that comparing with the surface morphology of BSE, BRM has no porous structure, so biodegradation agents could only act on the exterior of BRM. As a consequence, surface area of biodegraded raw material is significantly lower than surface area of biodegraded steam exploded raw material.

### SUMMARY

According to one aspect of the present invention, it is disclosed a composition derived from a naturally occurring ligno-cellulosic biomass comprising lignin and a total amount of carbohydrates having at least one carbohydrate. The composition is further characterized in that the BET surface area of the composition is in the range of 4 to 80 m²/g and the thermal decomposition of the composition via TGA shows a first derivative peak corresponding to a first lignin decomposition temperature range and a second derivative peak corresponding to a second lignin decomposition temperature range and the mass associated with the first derivative peak is greater than the mass associated with the second derivative peak. The Hydrogen content of the total amount of the carbohydrates is sufficient for deoxygenating the lignin in deoxygenating conditions.

According to another aspect of present invention, the first derivative peak has a maximum value corresponding to the first lignin decomposition temperature and the temperature corresponding to the maximum value of the first derivative peak is less than the temperature corresponding to the maximum value of a first derivative peak corresponding to a first lignin decomposition temperature range occurring in a thermal decomposition analysis of the naturally occurring ligno-cellulosic biomass used to derive the composition.

According to another aspect of the present invention, the maximum value of the first derivative peak is less than the maximum value of the first derivative peak corresponding to the first lignin decomposition temperature range occurring in a thermal decomposition analysis of the naturally occurring ligno-cellulosic biomass used to derive the composition by at least 20° C.

According to another aspect of the present invention, the weight of the total amount of total carbohydrates present in the composition is in the a range selected from the group consisting of 25 to 50%, 30 to 50%, 35 to 50%, 40 to 50%, 30 to 35%, 30 to 40%, 30 to 45% of the dry weight of the composition.

According to another aspect of the present invention, the amount of total lignin present in the composition is in the range of 30 to 80% of the dry weight of the composition and the weight percent of the carbohydrates plus the weight percent of the lignin is less than 100% of the dry weight of the composition.

According to another aspect of the present invention, the composition is void of ionic groups derived from mineral acids, organic acids and bases used in treating the naturally occurring ligno-cellulosic biomass.

According to another aspect of the present invention, the naturally occurring ligno-cellulosic biomass from which the composition was derived is selected from the group consisting of the grasses and food crops.

According to another aspect of the present invention, the composition is void of at least one enzyme which converts lignin.

According to another aspect of the present invention, the composition is made by
A) Soaking a ligno-cellulosic biomass feedstock in vapor or liquid water or mixture thereof in the temperature range of 100 to 210° C. for 1 minute to 24 hours to create a soaked biomass containing a dry content and a first liquid;
B) Separating at least a portion of the first liquid from the soaked biomass to create a first liquid stream and a first solid stream; wherein the first solid stream comprises the soaked biomass;
C) Steam exploding the first solid stream to create a steam exploded stream comprising solids and a second liquid,
D) Hydrolyzing the steam exploded stream in the presence of an enzyme or enzyme mixture to create a hydrolyzed stream comprised of carbohydrate monomers selected from the group consisting of glucose, xylose, and mannose.
E) Fermenting the hydrolyzed stream to create a fermented stream comprised of the composition and water, and
F) Separating at least a portion of the water from the fermented stream.

According to another aspect of the present invention, the enzyme or enzyme mixture has a glucans activity and the glucans activity is greater than zero and less than a value selected from the group consisting of 34, 30, 25, 20, 15, 12, 10, 7, and 5 FPU per gram of glucans in the steam exploded stream.

### BRIEF DESCRIPTION OF THE DRAWINGS

FIG. 1 is a TGA of naturally occurring Arundo donax.

FIG. 2 is a TGA of the composition derived from the Arundo donax of FIG. 1.

FIG. 3 is a TGA of naturally occurring wheat straw.

FIG. 4 is a TGA of the composition derived from the wheat straw of FIG. 3.

FIG. 5 is a TGA of naturally occurring corn stover.

FIG. 6 is a TGA of the composition derived from the corn stover of FIG. 5.

FIG. 7 is a TGA of the Arundo donax after pre-treatment.

### DESCRIPTION

This invention is to a composition derived from a naturally occurring ligno-cellulosic biomass comprising at least one carbohydrate and lignin having the unique decomposition temperatures and surface areas described below.

A natural or naturally occurring ligno-cellulosic biomass is the feed stock for this process. Ligno-cellulosic materials can be described as follows:

Apart from starch, the three major constituents in plant biomass are cellulose, hemicellulose and lignin, which are commonly referred to by the generic term lignocellulose. Polysaccharide-containing biomasses as a generic term include both starch and ligno-cellulosic biomasses. Therefore, some types of feedstocks can be plant biomass, polysaccharide containing biomass, and ligno-cellulosic biomass.

Polysaccharide-containing biomasses according to the present invention include any material containing polymeric sugars e.g. in the form of starch as well as refined starch, cellulose and hemicellulose.

Relevant types of naturally occurring biomasses for deriving the claimed invention may include biomasses derived from agricultural crops selected from the group consisting of starch containing grains, refined starch; corn stover, bagasse, straw e.g. from rice, wheat, rye, oat, barley, rape, sorghum; softwood e.g. Pinus sylvestris, Pinus radiate; hardwood e.g. Salix spp. Eucalyptus spp.; tuberse.g. beet, potato; cereals from e.g. rice, wheat, rye, oat, barley, rape, sorghum and corn; waste paper, fiber fractions from biogas processing, manure, residues from oil palm processing, municipal solid waste or the like. Although the experiments are limited to a few examples of the enumerated list above, the invention is believed applicable to all because the characterization is primarily to the unique characteristics of the lignin and surface area.

The ligno-cellulosic biomass feedstock used to derive the composition is preferably from the family usually called grasses. The proper name is the family known as Poaceae or Gramineae in the Class Liliopsida (the monocots) of the flowering plants. Plants of this family are usually called grasses, or, to distinguish them from other graminoids, true grasses. Bamboo is also included. There are about 600 genera and some 9,000-10,000 or more species of grasses (Kew Index of World Grass Species).

Poaceae includes the staple food grains and cereal crops grown around the world, lawn and forage grasses, and bamboo. Poaceae generally have hollow stems called culms, which are plugged (solid) at intervals called nodes, the points along the culm at which leaves arise. Grass leaves are usually alternate, distichous (in one plane) or rarely spiral, and parallel-veined. Each leaf is differentiated into a lower sheath which hugs the stem for a distance and a blade with margins usually entire. The leaf blades of many grasses are hardened with silica phytoliths, which helps discourage grazing animals. In some grasses (such as sword grass) this makes the edges of the grass blades sharp enough to cut human skin. A membranous appendage or fringe of hairs, called the ligule, lies at the junction between sheath and blade, preventing water or insects from penetrating into the sheath.

Grass blades grow at the base of the blade and not from elongated stem tips. This low growth point evolved in response to grazing animals and allows grasses to be grazed or mown regularly without severe damage to the plant.

Flowers of Poaceae are characteristically arranged in spikelets, each spikelet having one or more florets (the spikelets are further grouped into panicles or spikes). A spikelet consists of two (or sometimes fewer) bracts at the base, called glomes, followed by one or more florets. A floret consists of the flower surrounded by two bracts called the lemma (the external one) and the palea (the internal). The flowers are usually hermaphroditic (maize, monoecious, is an exception) and pollination is almost always anemophilous. The perianth is reduced to two scales, called lodicules, that expand and contract to spread the lemma and palea; these are generally interpreted to be modified sepals.

The fruit of Poaceae is a caryopsis in which the seed coat is fused to the fruit wall and thus, not separable from it (as in a maize kernel).

There are three general classifications of growth habit present in grasses; bunch-type (also called caespitose), stoloniferous and rhizomatous.

The success of the grasses lies in part in their morphology and growth processes, and in part in their physiological diversity. Most of the grasses divide into two physiological groups, using the C3 and C4 photosynthetic pathways for carbon fixation. The C4 grasses have a photosynthetic pathway linked to specialized Kranz leaf anatomy that particularly adapts them to hot climates and an atmosphere low in carbon dioxide.

C3 grasses are referred to as “cool season grasses” while C4 plants are considered “warm season grasses”. Grasses may be either annual or perennial. Examples of annual cool season are wheat, rye, annual bluegrass (annual meadowgrass, Poa annua and oat). Examples of perennial cool season are orchard grass (cocksfoot, Dactylis glomerata), fescue (Festuca spp), Kentucky Bluegrass and perennial ryegrass (Lolium perenne). Examples of annual warm season are corn, sudangrass and pearl millet. Examples of Perennial Warm Season are big bluestem, indiangrass, bermuda grass and switch grass.

One classification of the grass family recognizes twelve subfamilies: These are 1) anomochlooideae, a small lineage of broad-leaved grasses that includes two genera (Anomochloa, Streptochaeta); 2) Pharoideae, a small lineage of grasses that includes three genera, including Pharus and Leptaspis; 3) Puelioideae a small lineage that includes the African genus Puelia; 4) Pooideae which includes wheat, barley, oats, brome-grass (Bronnus) and reed-grasses (Calamagrostis); 5) Bambusoideae which includes bamboo; 6) Ehrhartoideae, which includes rice, and wild rice; 7) Arundinoideae, which includes the giant reed and common reed; 8) Centothecoideae, a small subfamily of 11 genera that is sometimes included in Panicoideae; 9) Chloridoideae including the lovegrasses (Eragrostis, ca. 350 species, including teff), dropseeds (Sporobolus, some 160 species), finger millet (Eleusine coracana (L.) Gaertn.), and the muhly grasses (Muhlenbergia, ca. 175 species); 10) Panicoideae including panic grass, maize, sorghum, sugar cane, most millets, fonio and bluestem grasses; 11) Micrairoideae and 12) Danthoniodieae including pampas grass; with Poa which is a genus of about 500 species of grasses, native to the temperate regions of both hemispheres.

Agricultural grasses grown for their edible seeds are called cereals. Three common cereals are rice, wheat and maize (corn). Of all crops, 70% are grasses.

Sugarcane is the major source of sugar production. Grasses are used for construction. Scaffolding made from bamboo is able to withstand typhoon force winds that would break steel scaffolding. Larger bamboos and Arundo donax have stout culms that can be used in a manner similar to timber, and grass roots stabilize the sod of sod houses. Arundo is used to make reeds for woodwind instruments, and bamboo is used for innumerable implements.

Another naturally ocurring ligno-cellulosic biomass feedstock may be woody plants or woods. A woody plant is a plant that uses wood as its structural tissue. These are typically perennial plants whose stems and larger roots are reinforced with wood produced adjacent to the vascular tissues. The main stem, larger branches, and roots of these plants are usually covered by a layer of thickened bark. Woody plants are usually either trees, shrubs, or lianas. Wood is a structural cellular adaptation that allows woody plants to grow from above ground stems year after year, thus making some woody plants the largest and tallest plants.

These plants need a vascular system to move water and nutrients from the roots to the leaves (xylem) and to move sugars from the leaves to the rest of the plant (phloem). There are two kinds of xylem: primary that is formed during primary growth from procambium and secondary xylem that is formed during secondary growth from vascular cambium.

What is usually called “wood” is the secondary xylem of such plants.

The two main groups in which secondary xylem can be found are:
1) conifers (Coniferae): there are some six hundred species of conifers. All species have secondary xylem, which is relatively uniform in structure throughout this group. Many conifers become tall trees: the secondary xylem of such trees is marketed as softwood.
2) angiosperms (Angiospermae): there are some quarter of a million to four hundred thousand species of angiosperms. Within this group secondary xylem has not been found in the monocots (e.g. Poaceae). Many non-monocot angiosperms become trees, and the secondary xylem of these is marketed as hardwood.

The term softwood is used to describe wood from trees that belong to gymnosperms. The gymnosperms are plants with naked seeds not enclosed in an ovary. These seed “fruits” are considered more primitive than hardwoods. Softwood trees are usually evergreen, hear cones, and have needles or scalelike leaves. They include conifer species e.g. pine, spruces, firs, and cedars. Wood hardness varies among the conifer species.

The term hardwood is used to describe wood from trees that belong to the angiosperm family Angiosperms are plants with ovules enclosed for protection in an ovary. When fertilized, these ovules develop into seeds. The hardwood trees are usually broad-leaved; in temperate and boreallatitudes they are mostly deciduous, but in tropics and subtropics mostly evergreen. These leaves can be either simple (single blades) or they can be compound with leaflets attached to a leaf stem. Although variable in shape all hardwood leaves have a distinct network of fine veins. The hardwood plants include e.g. Aspen, Birch, Cherry, Maple, Oak and Teak.

Therefore a preferred naturally occurring ligno-cellulosic biomass may be selected from the group consisting of the grasses and woods. Another preferred naturally occurring ligno-cellulosic biomass can be selected from the group consisting of the plants belonging to the conifers, angiosperms, Poaceae and families. Another preferred naturally occuring ligno-cellulosic biomass may be that biomass having at least 10% by weight of it dry matter as cellulose, or more preferably at least 5% by weight of its dry matter as cellulose.

The carbohydrate(s) comprising the invention is selected from the group of carbohydrates based upon the glucose, xylose, and mannose monomers.

The composition is derived from a naturally occurring ligno-cellulosic biomass through a process comprising the steps specified in the following description.

A pre-treatment is often used to ensure that the structure of the ligno-cellulosic content is rendered more accessible to the catalysts, such as enzymes, and at the same time the concentrations of harmful inhibitory by-products such as acetic acid, furfural and hydroxymethyl furfural remain substantially low. There are several strategies to achieve increased accessibility, many of which may yet be invented. The current strategies imply subjecting the ligno-cellulosic material to temperatures between 110-250° C. for 1-60 min e.g.:

Hot water extraction

Multistage dilute acid hydrolysis, which removes dissolved material before inhibitory substances are formed

Dilute acid hydrolyses at relatively low severity conditions

Alkaline wet oxidation

Steam explosion.

A preferred pretreatment of a naturally occurring ligno-cellulosic biomass includes a soaking of the naturally occurring ligno-cellulosic biomass feedstock and a steam explosion of at least a part of the soaked naturally occurring ligno-cellulosic biomass feedstock.

The soaking occurs in a substance such as water in either vapor form, steam, or liquid form or liquid and steam together, to produce a product. The product is a soaked biomass containing a first liquid, with the first liquid usually being water in its liquid or vapor form or some mixture.

This soaking can be done by any number of techniques that expose a substance to water, which could be steam or liquid or mixture of steam and water, or, more in general, to water at high temperature and high pressure. The temperature should be in one of the following ranges: 145 to 165° C., 120 to 210° C., 140 to 210° C., 150 to 200° C., 155 to 185° C., 160 to 180° C. Although the time could be lengthy, such as up to but less than 24 hours, or less than 16 hours, or less than 12 hours, or less than 9 hours, or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

If steam is used, it is preferably saturated, but could be superheated. The soaking step can be batch or continuous, with or without stirring. A low temperature soak prior to the high temperature soak can be used. The temperature of the low temperature soak is in the range of 25 to 90° C. Although the time could be lengthy, such as up to but less than 24 hours, or less than 16 hours, or less than 12 hours, or less than 9 hours or less than 6 hours; the time of exposure is preferably quite short, ranging from 1 minute to 6 hours, from 1 minute to 4 hours, from 1 minute to 3 hours, from 1 minute to 2.5 hours, more preferably 5 minutes to 1.5 hours, 5 minutes to 1 hour, 15 minutes to 1 hour.

Either soaking step could also include the addition of other compounds, e.g. H₂SO4, NH₃, in order to achieve higher performance later on in the process.

The product comprising the first liquid is then passed to a separation step where the first liquid is separated from the soaked biomass. The liquid will not completely separate so that at least a portion of the liquid is separated, with preferably as much liquid as possible in an economic time frame. The liquid from this separation step is known as the first liquid stream comprising the first liquid. The first liquid will be the liquid used in the soaking, generally water and the soluble species of the feedstock. These water soluble species are glucan, xylan, galactan, arabinan, glucolygomers, xyloolygomers, galactolygomers and arabinolygomers. The solid biomass is called the first solid stream as it contains most, if not all, of the solids.

The separation of the liquid can again be done by known techniques and likely some which have yet to be invented. A preferred piece of equipment is a press, as a press will generate a liquid under high pressure.

The first solid stream is then steam exploded to create a steam exploded stream, comprising solids and a second liquid. Steam explosion is a well known technique in the biomass field and any of the systems available today and in the future are believed suitable for this step. The severity of the steam explosion is known in the literature as Ro, and is a function of time and temperature and is expressed as

Ro=texp[(T−100)/14.75]

with temperature, T expressed in Celsius and time, t, expressed in common units.

The formula is also expressed as Log(Ro), namely

Log(Ro)=Ln(t)+[(T−100)/14.75].

Log(Ro) is preferably in the ranges of 2.8 to 5.3, 3 to 5.3, 3 to 5.0 and 3 to 4.3.

The steam exploded stream may be optionally washed at least with water and there may be other additives used as well. It is conceivable that another liquid may be used in the future, so water is not believed to be absolutely essential. At this point, water is the preferred liquid and if water is used, it is considered the third liquid. The liquid effluent from the optional wash is the third liquid stream. This wash step is not considered essential and is optional.

The washed exploded stream is then processed to remove at least a portion of the liquid in the washed exploded material. This separation step is also optional. The term at least a portion is removed, is to remind one that while removal of as much liquid as possible is desirable (pressing), it is unlikely that 100% removal is possible. In any event, 100% removal of the water is not desirable since water is needed for the subsequent hydrolysis reaction. The preferred process for this step is again a press, but other known techniques and those not invented yet are believed to be suitable. The products separated from this process are solids in the second solid stream and liquids in the second liquid stream.

The steam exploded stream is then subjected to hydrolysis to create a hydrolyzed stream. Optionally at least a part of the liquid of the first liquid stream is added to the steam exploded stream. Also, water is optionally added. Hydrolysis of the steam exploded stream is realized by contacting the steam exploded stream with a catalyst. Enzymes and enzyme composition, or enzyme mixture, is the preferred catalyst. While the use of laccase, an enzyme known to alter lignin, may be used, the composition is preferably void of at least one enzyme which converts lignin. A preferred hydrolysis of the steam exploded stream comprises the step of:

A) Contacting the steam exploded stream with at least a portion of a solvent, the solvent comprised of water soluble hydrolyzed species; wherein at least some of the water soluble hydrolyzed species are the same as the water soluble hydrolyzed species obtainable from the hydrolysis of the steam exploded stream;

B) maintaining the contact between the steam exploded stream and the solvent at a temperature in the range of 20° C. to 200° C. for a time in the range of 5 minutes to 72 hours to create a hydrolyzed stream from the steam exploded stream.

The hydrolyzed stream is comprised of carbohydrate monomers selected from the group consisting of glucose, xylose, and mannose.

The hydrolyzed stream is subjected to fermentation to create a fermented stream comprised of the composition and water. The fermentation is performed by means of addition of yeast or yeast composition to the hydrolyzed stream.

Eventually hydrolysis and fermentation can be performed simultaneously, according to the well known technique of simultaneous saccharification and fermentation (SSF).

The composition derived from naturally occurring ligno-cellulosic biomass is separated from the water in the fermented stream. The separation of the liquid can be done by known techniques and likely some which have yet to be invented. A preferred piece of equipment is a press.

Preferably, the enzymatic hydrolysis is conducted in the presence of a low dosage of the enzymes or enzyme mixture. The preferred strategy in the art is to hydrolyze as much carbohydrates as possible in the pre-treated stream; this requires the use of a high dosage of enzymes or enzyme mixture, which may be not economically convenient.

As well known in the art, the dosage of an enzyme or enzyme mixture may be expresses in terms of the activity of the enzyme or enzyme mixture on standard substrates. Filter Paper Unit (FPU) is the main parameter used for measuring the activity of an enzyme or enzyme mixture on glucans. Preferably the enzyme or enzyme mixture has a glucans activity and the glucans activity is greater than zero and less than a value selected from the group consisting of 34, 30, 25, 20, 15, 12, 10, 7, and 5 FPU per gram of glucans in the steam exploded stream.

Inventors have found that, while removing more carbohydrates from the pre-treated stream by using higher enzyme dosage may benot economically convenient, the carbohydrates left in the composition may be usefully converted to other products in following conversion process, preferably by means of thermo-chemical conversion method. Thereby, the disclosed composition is useful as an intermediate product in a biorefinery scheme, wherein the components of the naturally occurring ligno-cellulosic biomass are converted to different products in different conversion steps.

It is known in the art that Hydrogen may be produced from carbohydrates, for instance by means of thermo-chemical processes. The amount of Hydrogen which may be produced from the carbohydrates in the disclosed composition is sufficient for deoxygenating the lignin in the composition. Any method known in the art, and still to be invented, may be used for producing Hydrogen from the carbohydrates of the composition. An example of a method which may be used for converting carbohydrates into Hydrogen is disclosed in Guodong Wen et al., “Direct conversion of cellulose into hydrogen by aqueous-phase reforming process”, Catalysis Communications 11 (2010) 522-526.

By the expression “deoxygenating the lignin” it is meant that at least 99% of the Oxygen in the lignin is removed from the lignin. In the process of deoxygenating the lignin one or more liquid products may be produced, in one or more conversion steps. The liquid products are in a liquid state at a pressure of 1 bar and at a temperature of 25° C., and may comprise, for instance, benzene, toluene, o-, m- and p-xylenes, heptadecane, ethylcyclohexane, propyl benzene, ethyl benzene. Other products in gas state, some of them comprising oxygen, may be produced in deoxygenating the lignin. A review of methods which can used for deoxygenating lignin is contained in Joseph Zakzeski et al., “The Catalytic Valorization of Lignin for the Production of Renewable chemicals”, Chem. Rev. 2010, 110, 3552-3599.

The composition is different from naturally occurring ligno-cellulosic biomass in that it has a large surface area as measured by BET. BET is a standard technique for measuring surface area of porous materials. Measurements were performed by means of an automatic porosimeter. Micromeritics Mod. ASAP 2010. Samples were dried in an oven at 120° C. for 12 hours. Surface area values were calculated according to the standard Brunauer, Emmett and Teller (BET) method.

The BET surface area of the dry composition is in the range of 4 to 80 m²/gm, with 4 to 50 m²/gm being more preferable, 4 to 25 m²/gm being even more preferred, and 4 to 15 m²/gm being even more preferred and 4 to 12 m²/gm being the most preferred. The surface area of the claimed compositions are disclosed in Table 1.

The composition is further characterized by the peaks generated during a thermal gravimetric analysis, known as TGA.

TGA is a widely used technique for studying decomposition of a solid or liquid material, due to the effect of temperature. In a TGA, a sample of the material is subjected to a thermal ramp from an initial temperature to a final temperature in a certain gas atmosphere and the weight is recorded. Weight losses of the material are due to thermal decomposition, in which a part of the sample is transformed from solid or liquid phase to vapour phase. If the material is a composition of many components, each component can decompose at a specific temperature or in a specific temperature range.

In thermogravimetric analysis, the plot of the weight with respect to temperature and the plot of the first derivative of weight with respect to temperature are commonly used.

If the decomposition of the material or of a component of the material occur in a specific range of temperature, the plot of the first derivative of weight with respect to temperature presents a maximum in the specific range of temperature, defined also as first derivative peak. The value of temperature corresponding to the first derivative peak is considered the decomposition temperature of the material or of that component of the material.

If the material is a composition of many components, which decompose in different specific temperature ranges, the plot of the first derivative of weight with respect to temperature presents first derivative peaks associated to the decomposition of each component in each specific temperature range. The temperature values corresponding to the first derivative peaks are considered the decomposition temperatures of each component of the material.

As a general rule, a maximum is located between two minima. The values of temperature corresponding to the minima are considered as the initial decomposition temperature and the final decomposition temperature of the decomposition temperature range of the component whose decomposition temperature corresponds to the first derivative peak comprised between the two minima. In this way, a derivative peak corresponds to decomposition temperature range. The weight loss of the material in the range between the initial decomposition temperature and the final decomposition temperature is associated to the decomposition of that component of the material and to the first derivative peak.

The TGA analysis was conducted on a TA Q series Instrument: TGA Q500 SW 6.4.193.

Sample weight was in the range at 10-20 mg, referred to dry weight.

Drying procedure of 48 hrs at 40° C. was optionally applied.

Samples were sieved below 20 Mesh by means of a Wiley Mini-Mill.

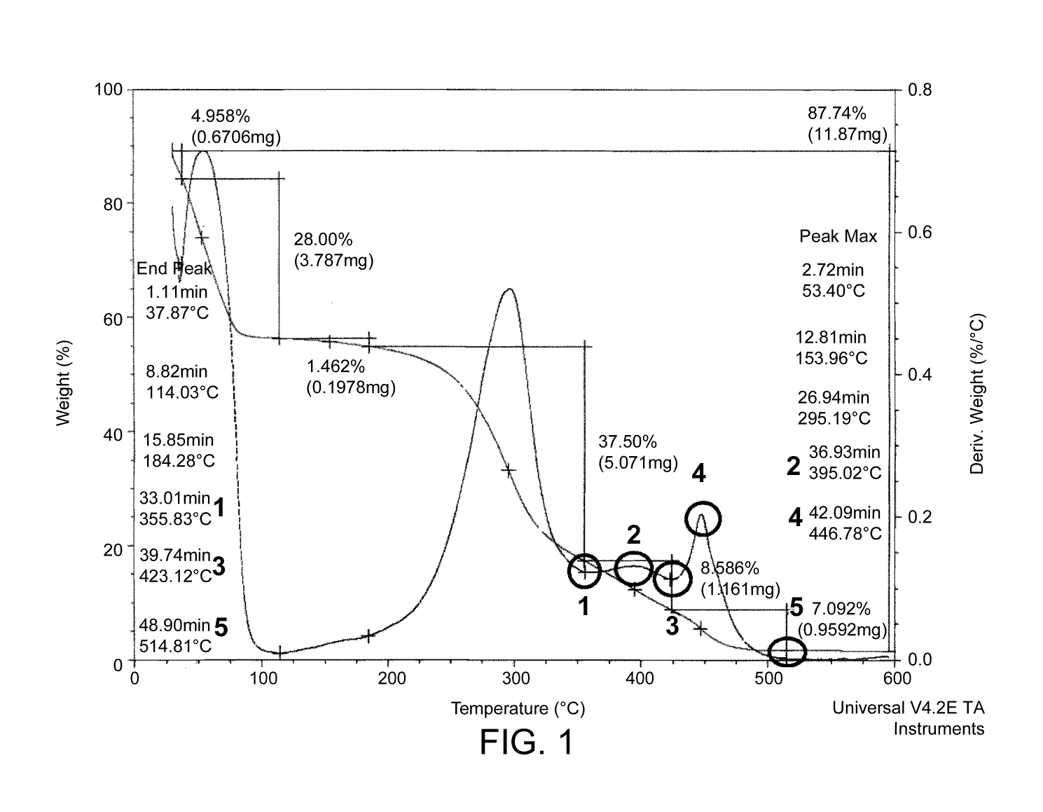

Measurements were conducted run in air at 10° C./min, at an air flow of 60 mL/min, in the range of temperature from 30° C. to 600° C. The thermal decomposition characterization can be explained by referring to the Figures. FIG. 1 is a TGA chart of naturally occurring Arundo donax.

The TGA of FIG. 1 displays two lines. One is the weight percent of the sample decomposing as a function of the temperature. The other line is the derivative of the first line. It is the derivative line which is analyzed. Starting from the left side of the Figure, there is a peak corresponding which ends at 37.87° C. and another peak corresponding to 38.87° C. to 114.03° C. These two peaks correspond to the loss of water and other volatiles which occur in a small amount. In this case, 32.958% of the sample is water and volatiles removed at less than 114.03° C.

There is also a peak ending at 184.28° C. which is of little analytic value.

The next peak which has a maximum value greater than 250° C. and less than 325° C. (295.19° C.) corresponds to the decomposition of the carbohydrates present in the composition.

The circle labeled 1 marks the start of the temperature range of the first lignin decomposition temperature and begins at the end of the carbohydrate peak (355.83° C.) and ends at the data point labeled 3 (423.12° C.). This peak has a maximum value corresponding to a first lignin decomposition temperature of 395.02° C. (Labeled 2).

There is a second peak corresponding to a second lignin decomposition temperature ranging from 423.12° C. (Labeled 3) to 514.81° C. (Labeled 5). Label 4 marks the maximum of the second lignin decomposition temperature at 446.78° C. Each peak has a mass associated with it. In the case of the first peak, 1.161 mg was decomposed in the first temperature range and 0.959 gm decomposed in the second temperature range.

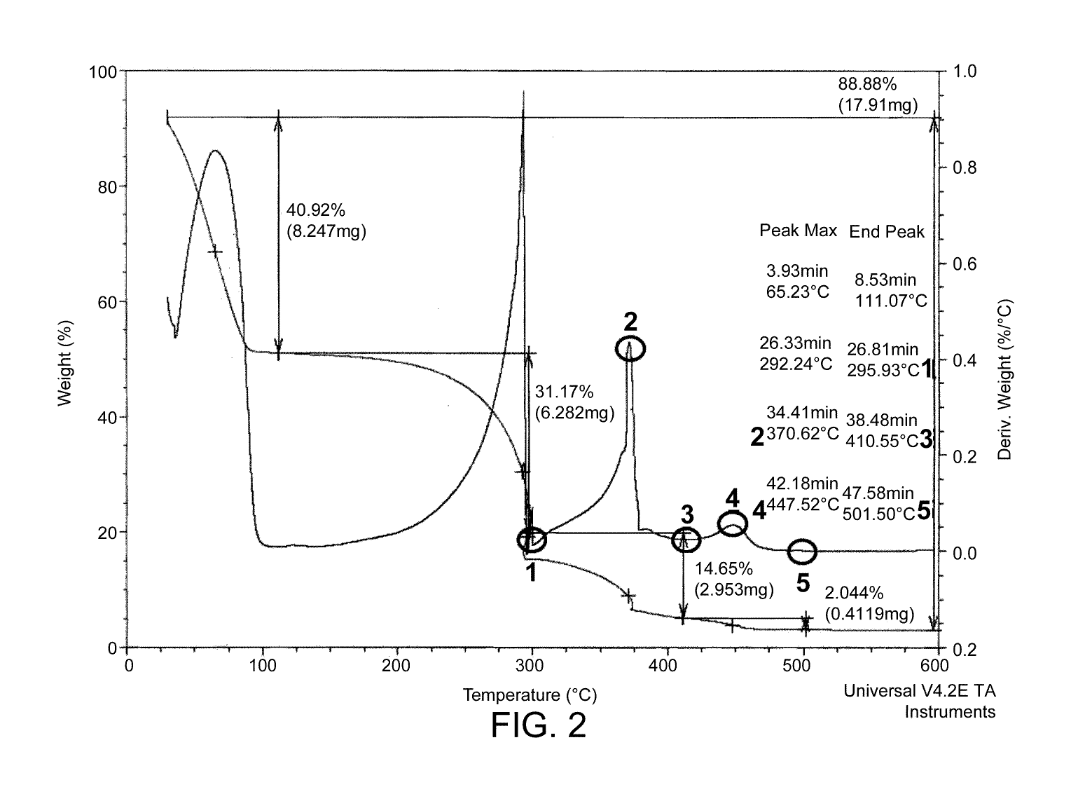

FIG. 2 is a TGA chart of the claimed composition derived from the naturally occurring Arundo donax of FIG. 1. The first temperature range of the first lignin decomposition temperature is in the range of 295.93 to 410.55° C. (Labels 1 and 3), with the maximum occurring at 370.62° C. (Label 2). The second peak corresponding to the second lignin decomposition temperature range is between 410.55° C. to 501.5° C. (Labels 3 and 5), with a maximum occurring at 447.52° C. (Label 4).

Should the naturally occurring ligno-cellulosic biomass used to derive the lignin composition be a mixture of different species of grasses or plants or other materials, then the mixture of the naturally occurring ligno-cellulosic biomass is what should be used for the comparison with the material from which the composition was derived.

The composition created has the characteristics that temperature corresponding to the maximum value of the first lignin decomposition peak is less than the temperature corresponding to the maximum value of the first lignin decomposition peak of the naturally occurring ligno-cellulosic biomass. This difference is marked and unexpected, with the maximum value of the first lignin decomposition peak being less than the temperature corresponding to the maximum value of the first lignin decomposition peak of the naturally occurring ligno-cellulosic biomass by a value selected from the group consisting of at least 10° C., at least 15° C., at least 20° C., and at least 25° C.

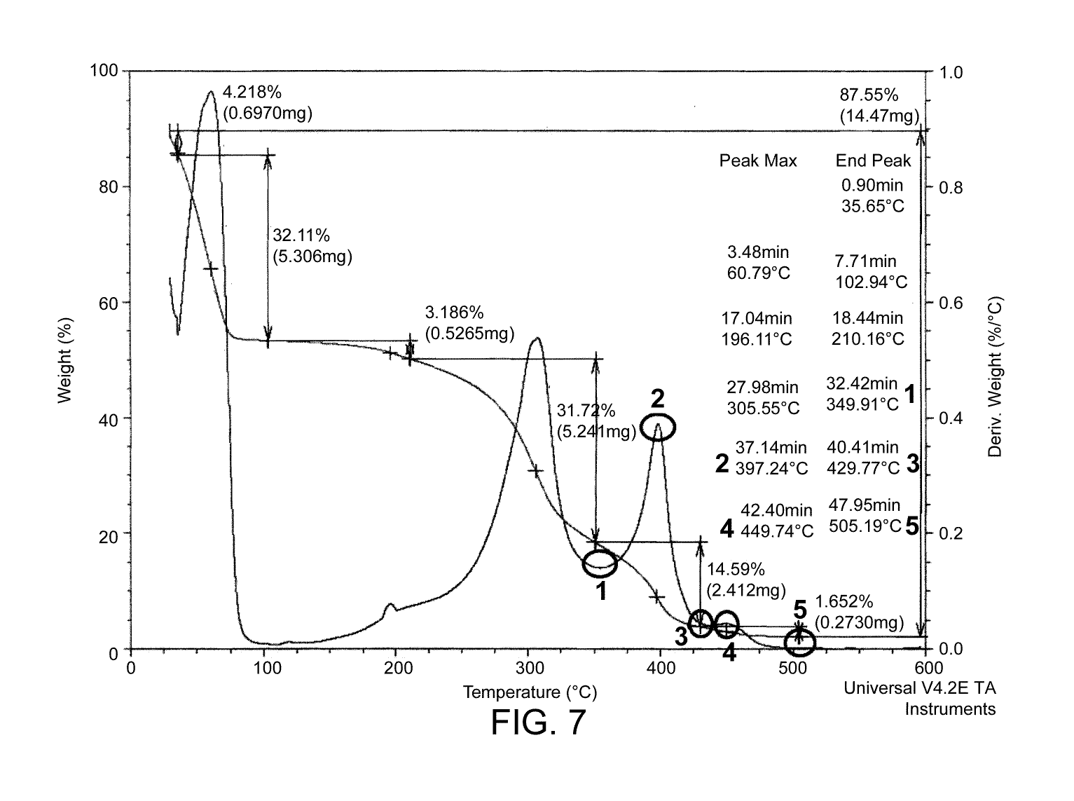

This reduction in the maximum value of the first lignin decomposition temperature can be compared to the maximum value of the first lignin decomposition temperature after pre-treatment. As shown in FIG. 7, for Arundo donax, the pre-treatment of soaking and steam explosion does not reduce the maximum value of the first lignin decomposition temperature.

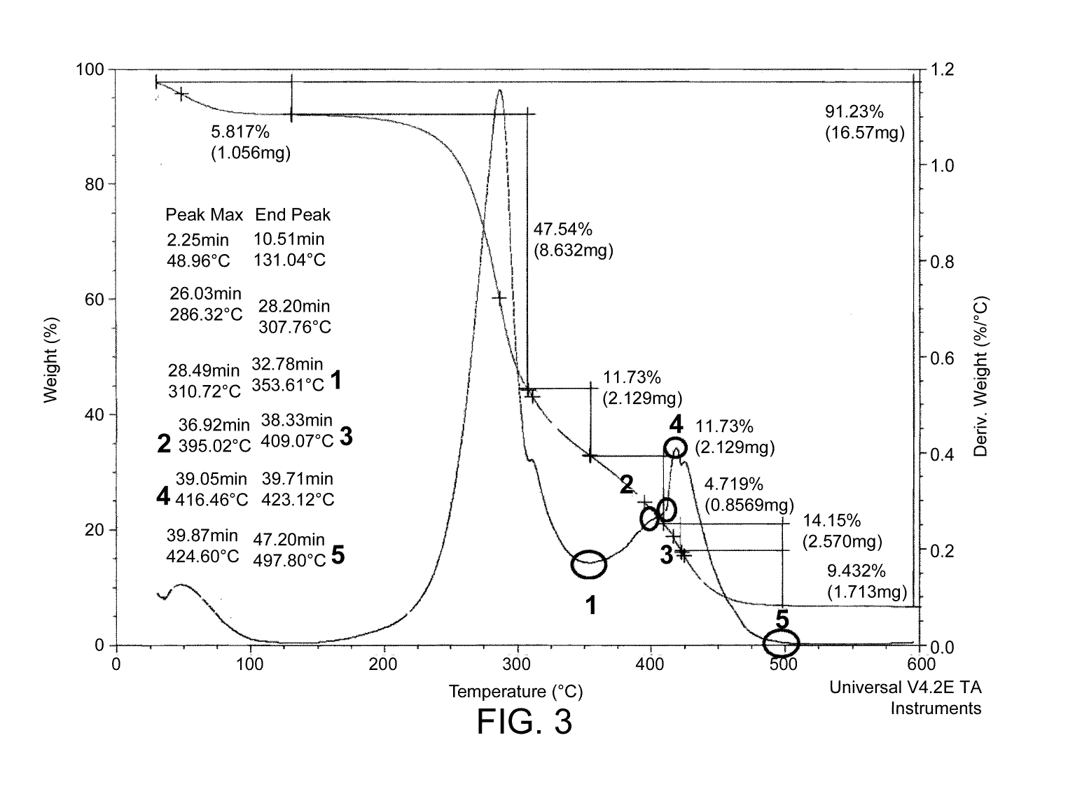

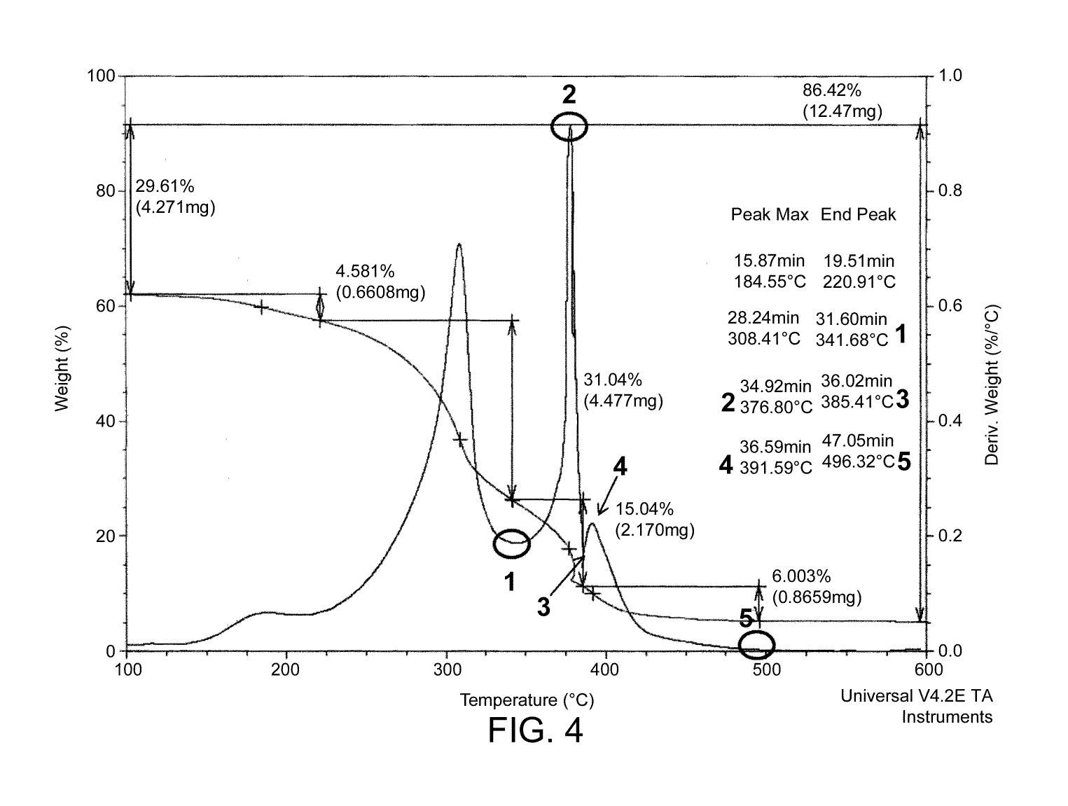

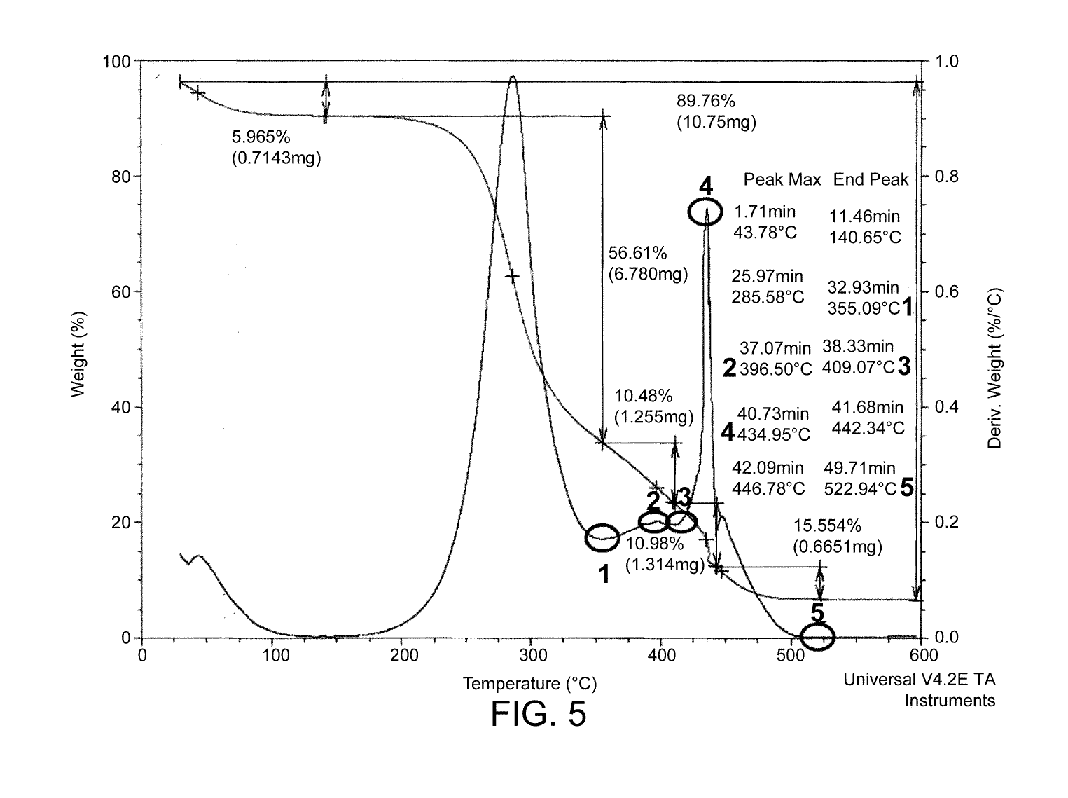

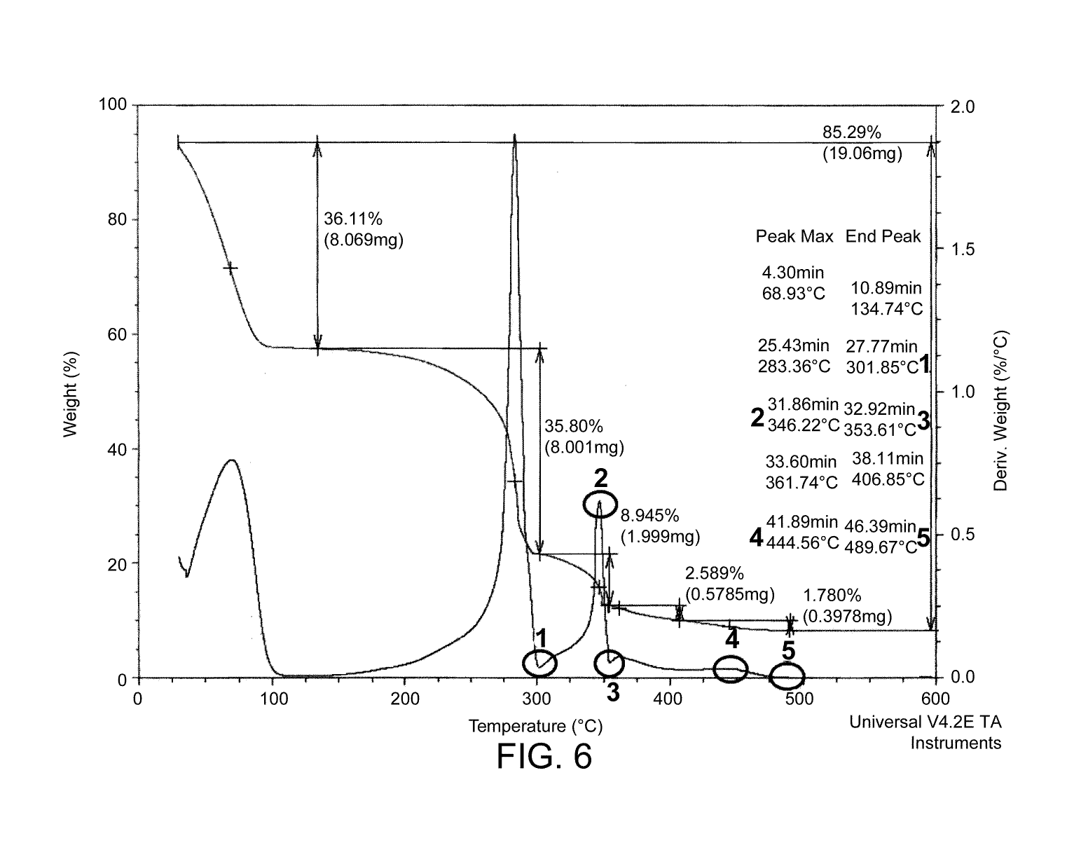

FIGS. 3 to 6 are comparable TGA analyses for wheat straw and corn stover, all demonstrating the thermal characteristics. The results of the analysis is compiled in Table 1. As can be seen in Table 2, when the lignin decomposition temperature range has several shoulders or a tail such as that associated with lignin, the second decomposition range includes those peaks/tail as well. The various small amounts are totaled to reach the second decomposition amount released.

Additionally, the absolute mass on a dry basis associated with the first lignin decomposition peak of the claimed lignin composition is greater than the absolute mass on a dry basis of the second lignin decomposition peak. While for Arundo donax, the absolute mass of the first decomposition temperature of the naturally occurring ligno-cellulosic biomass is greater than the absolute mass of the second decomposition temperature of the naturally occurring ligno-cellulosic biomass, this is not true for many ligno-cellulosic biomasses such as corn stover and wheat straw. However, after conversion, the lignin composition derived from these biomasses has a mass on a dry basis associated with the first lignin decomposition temperature that is greater than the mass on a dry basis associated with the second lignin decomposition temperature.

It is noted that the claimed composition can be further characterized by comparing the temperature associated with the maximum value of the first lignin decomposition range with the temperature associated with the maximum value of the first lignin decomposition range of the ligno-cellulosic biomass used to derive the claimed composition. It is noted by comparing FIGS. 2 and 1, that the first peak of FIG. 2 has a maximum value corresponding to a temperature of 371° C. and the temperature corresponding to the maximum value of the first peak is less than the temperature of 395° C. corresponding to the maximum value of a first peak corresponding to a first lignin decomposition temperature range occurring in a thermal decomposition analysis of the naturally occurring ligno-cellulosic biomass used to derive the composition.

The composition can be further characterized by the relative amount of carbohydrates present on a dry basis. Preferably, the weight of the total amount of total carbohydrates present in the composition is in the a range selected from the group consisting of of 0.25 to 50%, 30 to 50%, 35 to 50%, 40 to 50%, 30 to 35%, 30 to 40%, 30 to 45% of the dry weight of the composition.

The composition can be further characterized by the relative amount of lignin present on a dry basis. Preferably, the amount of total lignin present in the composition is in the range of 30 to 80% of the dry weight of the composition and the weight percent of the carbohydrates plus the weight percent of the lignin is less than 100% of the dry weight of the composition.

The composition is void of ionic groups derived from mineral acids, organic acids and bases used in treating the naturally occurring ligno-cellulosic biomass. If present, ionic groups are produced from the naturally occurring ligno-cellulosic biomass in the process for obtaining the disclosed composition.

Because the claimed composition may vary with the starting material from which it is derived, the naturally occurring ligno-cellulosic biomass from which the composition was derived is selected from the group consisting of the grasses and food crops.

The representative preparation of the composition is as follows: First, naturally occurring ligno-cellulosic biomass was inserted into a reactor and subjected to a hydrothermal treatment at a temperature of 155° C. for a time of 155 minutes. Products of hydrothermal treatment were separated into a liquid stream and a solid stream by means of a pressing system.

The solid stream was subjected to steam explosion at a temperature of 195° C. for 4 minutes. Steam exploded products are referred to as pretreated ligno-cellulosic biomass, shown in FIG. 7. The liquid stream and steam exploded products were mixed together and water was added until reaching a mixture containing 15% of dry matter.

The samples was then hydrolyzed enzymatically by adding the enzymatic cocktail Ctec2 by Novozyme to the mixture to obtain an enzyme cocktail concentration of 10 mg of proteins per gram of cellulose. Enzymatic hydrolysis was conducted at a pH of 5 and at a temperature of 50° C. for 24 hours and a hydrolyzed stream was generated.

The hydrolyzed stream was subjected to fermentation by inoculation of yeast RN1016 at a concentration of 0.5 g/Kg of hydrolyzed stream and the addiction of 3 g of urea per Kg of hydrolyzed stream. Fermentation was performed at a temperature of 32° C. and a pH of 5 for a time of 72 hours and a fermented stream was obtained.

Products of fermentation were removed from the fermented stream by means of thermal evaporation at a temperature of 70° C. for a time of 72 hours and fermentation residues were obtained. The fermentation residues were pressed to separate a liquid fraction and the solid composition derived from lignocellulosic biomass having the properties claimed in this application.

TABLE 1

| | | Carbohydrates | Lignin content |
|---|---|---|---|
| | Surface area | (g/100 g | (g/100 g |
| | (m2/g) | Dry Matter) | Dry Matter) |
| Composition derived | 5.33 | 38.8 | 57.0 |
| from Arundo Donax | | | |
| Composition derived | 7.57 | 25.0 | 42.5 |
| from Wheat Straw | | | |

TABLE 2

| | | | | | Mass | |
|---|---|---|---|---|---|---|
| | | | | | released | |
| | | Second | | | in the first | Mass released in |
| | First peak | peak | First | Second | temperature | the second |
| | temperature | temperature | temperature | temperature | range | temperature |
| | (° C.) | (° C.) | range (° C.) | range (° C.) | (mg) | range (mg) |
| Naturallyoccurring | 395 | 447 | 356-423 | 423-515 | 1.161 | 0.9592 |
| Arundo | | | | | | |
| Donax | | | | | | |
| Pretreated | 397 | 450 | 350-430 | 430-505 | 2.412 | 0.2730 |
| Arundo | | | | | | |
| Donax | | | | | | |
| Composition | 370 | 448 | 296-411 | 411-502 | 2.953 | 0.4119 |
| derived from | | | | | | |
| Arundo | | | | | | |
| Donax | | | | | | |
| Naturally | 395 | 416 | 354-409 | 409-498 | 2.129 | 5.1399 |
| occurring | | | | | | (0.8569 + 2.570 + 1.713) |
| Wheat Straw | | | | | | |
| Composition | 377 | 392 | 342-385 | 385-496 | 2.170 | 0.8659 |
| derived from | | | | | | |
| Wheat Straw | | | | | | |
| Naturally | 397 | 435 | 355-409 | 409-523 | 1.225 | 1.9791 |
| occurring | | | | | | (1.314 + 0.6651) |
| Corn Stover | | | | | | |
| Composition | 346 | 445 | 302-354 | 354-490 | 1.999 | 0.9763 |
| derived from | | | | | | (0.5785 + 0.3978) |
| Corn stover | | | | | | |

We claim:

1-10. (canceled)

11. A composition derived from a naturally occurring ligno-cellulosic biomass comprising lignin and a total amount of carbohydrates having at least one carbohydrate characterized in that the BET surface area of the composition is in the range of 4 to 80 m²/g and the thermal decomposition of the composition via TGA shows a first derivative peak corresponding to a first lignin decomposition temperature range and a second derivative peak corresponding to a second lignin decomposition temperature range and the mass associated with the first derivative peak is greater than the mass associated with the second derivative peak, wherein the Hydrogen content of the total amount of the carbohydrates is sufficient for deoxygenating the lignin in deoxygenating conditions.

12. The composition of claim 11, wherein the first derivative peak has a maximum value corresponding to the first lignin decomposition temperature and the temperature corresponding to the maximum value of the first derivative peak is less than the temperature corresponding to the maximum value of a first derivative peak corresponding to a first lignin decomposition temperature range occurring in a thermal decomposition analysis of the naturally occurring ligno-cellulosic biomass used to derive the composition.

13. The composition of claim 12, wherein the maximum value of the first derivative peak is less than the maximum value of the first derivative peak corresponding to the first lignin decomposition temperature range occurring in a thermal decomposition analysis of the naturally occurring ligno-cellulosic biomass used to derive the composition by at least 20° C.

14. The composition according to claim 11, wherein the weight of the total amount of carbohydrates present in the composition is in a range selected from the group consisting of 25 to 50%, 30 to 50%, 40 to 50%, 30 to 35%, 30 to 40%, 30 to 45% of the dry weight of the composition.

15. The composition according to claim 11, wherein the amount of total lignin present in the composition is in the range of 30 to 80% of the dry weight of the composition and the weight percent of the carbohydrates plus the weight percent of the lignin is less than 100% of the dry weight of the composition.

16. The composition according to claim 11, wherein the composition is void of ionic groups derived from mineral acids, organic acids and bases used in treating the naturally occurring ligno-cellulosic biomass.

17. The composition of claim 11, wherein the naturally occurring ligno-cellulosic biomass from which the composition was derived is selected from the group consisting of grasses and food crops.

18. The composition of claim 11, wherein the composition is void of at least one enzyme which converts lignin.

19. The composition of claim 11, wherein the composition is made by a process comprising:
A) Soaking a ligno-cellulosic biomass feedstock in vapor or liquid water or mixture thereof in the temperature range of 100 to 210° C. for 1 minute to 24 hours to create a soaked biomass containing a dry content and a first liquid;
B) Separating at least a portion of the first liquid from the soaked biomass to create a first liquid stream and a first solid stream; wherein the first solid stream comprises the soaked biomass;
C) Steam exploding the first solid stream to create a steam exploded stream comprising solids and a second liquid;
D) Hydrolyzing the steam exploded stream in the presence of an enzyme or enzyme mixture to create a hydrolyzed stream comprised of carbohydrate monomers selected from the group consisting of glucose, xylose, and mannose;
E) Fermenting the hydrolyzed stream to create a fermented stream comprised of the composition and water; and
F) Separating at least a portion of the water from the fermented stream.

20. The composition of claim 19, wherein the enzyme or enzyme mixture has a glucans activity and the glucans activity is greater than zero and less than a value selected from the group consisting of 34, 30, 25, 20, 15, 12, 10, 7, and 5 FPU per gram of glucans in the steam exploded stream.
